(19) United States
(12) Patent Application Publication
(10) Pub. No.: US 2002/0132359 A1
(43) Pub. Date: Sep. 19, 2002

(54) Dispensing unit for oxygen-sensitive drugs

(76) Inventor: Kenneth C. Waterman, East Lyme, CT (US)

Correspondence Address: Gregg C. Benson, Pfizer Inc., Patent Department, MS 4159, Eastern Point Road, Groton, CT, 06340, US

(21) Appl. No.: 10/099,646

(22) Filed: Mar. 15, 2002

Related U.S. Application Data
(60) Provisional application No. 60/276,685, filed on Mar. 16, 2001.

(51) Int. Cl. G01N 33/00

(52) U.S. Cl. 436/127; 436/136; 436/138; 435/287.6; 422/58; 422/99; 422/102

(57) ABSTRACT

A means for dispensing a single unit dose of an oxygen-sensitive drug without exposing the remaining unit dosages to oxygen is described herein. Each unit dose is individually encapsulated in the pharmaceutical packaging construction such that when one unit dose is dispensed the other unit doses remain encapsulated. An oxygen-absorber is also incorporated into the construction such that the oxygen absorber has sufficient contact with the air surrounding the oxygen-sensitive drug to remove at least a portion of the oxygen in the air to reduce or eliminate undesirable oxidative degradation of the drug in its encapsulated environment.

This application claims the benefit of U.S. Provisional Patent Application No. 60/276,685, filed Mar. 16, 2001, incorporated in its entirety herein by reference.

### FIELD OF THE INVENTION

The present invention relates to a means for dispensing a single unit dose of an oxygen-sensitive drug without exposing the remaining unit dosages to oxygen, in particular, a pharmaceutical packaging construction having an oxygen-absorber incorporated therein.

### BACKGROUND

Oxygen induced drug degradation often limits shelf-life (expiration date) or may render a drug unmarketable. In fact, drug candidates that are highly oxygen sensitive are often excluded from further development. In a number of cases, oxygen sensitivity occurs only in the presence of certain excipients. Since oxidation is often not accelerated by standard Arrhenius based increased temperature studies (i.e., accelerated aging studies), there are a number of drug candidates where the oxygen sensitivity of the drug is not recognized until drug development has progressed into late stages of development at which time a significant amount of resources have been expended. At the later stages of development, reformulation and addition of standard antioxidants can require considerably more time and money. Changes in formulation may also require re-evaluation of clinical data. Therefore, there is a need for a means of reducing or eliminating oxygen based drug instability without requiring a formulation change.

Even in early drug development, there is a need for oxidation prevention with a new drug candidate to provide adequate stability for initial studies without investing a lot of resources prior to proof of concept. Once a candidate has been selected for further development, the oxygen-sensitivity can then be preferably addressed at the earlier stage of development.

Single unit dose packaging provides several advantages in the pharmaceutical field. In some countries, single unit dose packaging provides a regulatory approved method for pharmacy dispensing of the drug. For example, in Europe the majority of prescription pharmaceuticals are dispensed in blister packaging. Unit dose packaging can be a valuable method for assuring patient compliance with a dosing regimen. Such packaging can also prevent exposure of individual dosages to the environment in contrast to bottle packaging where once the bottle is opened, it is difficult to assure resealing of the bottle. There are also marketing considerations which can make single unit packaging desirable.

Blister packaging can show various degrees of oxygen permeability. The most impermeable packaging consists of using foil for both the blister and the lid. This packaging leads to an opaque blister, which can be less desirable from a marketing consideration. Moreover, the foil-foil blister must be packaged in an anaerobic environment to assure there is no oxygen in the headspace. In practical terms, the oxygen level left in the headspace is often above 5%, and rarely down to 0.1%, due to the oxygen on the dosage form as well as in the headspace. It would therefore be desirable to provide a method for removing oxygen to still lower levels in a blister packaging, without resorting to extraordinary and expensive manufacturing techniques.

Although a variety of oxygen removal techniques are well known in the food industry, there is much less known about oxygen removal for pharmaceutical applications and no mention of using oxygen absorbers in single unit packaging. In the pharmaceutical industry, there have been some limited reports of using oxygen absorbers to stabilize drugs. For example, in 1984, tablets of an anti-inflammatory drug were stabilized in large glass jars with oxygen absorbing sachets for six months at 50° C. (Japanese Patent No. SHO59-176247). The source of the oxygen being removed is primarily from the headspace and not from ingress. Similarly, Japanese Patent No. 96-253638 describes cold remedy powders stabilized in impermeable bottles by either nitrogen purging or with oxygen absorbers in the bottle. In a 1990 publication, it was reported that L-cysteine in an ophthalmic ointment was stored with an oxygen absorber. (See, i.e., Kyushu Yakugakkai Kaiho, “L-Cysteine Ophthalmic Solution Stabilized with Oxygen Absorber,” 44, 37-41 (1990).) In 1995, it was reported that tonic solutions of vitamin C were stabilized using a bottle cap having an oxygen absorber covered with a polyolefin (Japanese Patent No. SHO94-17056). U.S. Pat. No. 5,839,593 describes the incorporation of an oxygen-absorber into the liner of a bottle cap. More recently, U.S. Pat. Nos. 6,093,572; 6,007,529; and 5,881,534; and PCT publication WO 9737628 describe the use of oxygen absorbers with parenterals and their particular benefit for sterilization. Placement of oxygen-absorbing sachets between an intravenous (IV) bag or blood bag and its outer packaging is commonly used in commercial applications. Pre-filled syringes with absorbers between the syringes and outer packaging are also known.

In spite of the wide use of oxygen absorbers in the food industry and more limited reports in the pharmaceutical industry, there is no information or guidance as to the appropriateness of this technology or best practice methods for use with solid dosage form pharmaceuticals. In particular, there is no information with respect to the efficacy of oxygen absorbers in pharmaceutical packaging using a drug that has a high sensitivity to oxygen. Unlike prior reports where solid dosage forms are stored in glass, there is no reported use of oxygen absorbers with highly permeable plastic packaging for pharmaceutical applications. In addition, there is no information describing relatively low moisture conditions to minimize physical problems (e.g., tablet sticking, disintegration, or dissolution) and chemical stability issues (e.g., hydrolysis). In particular, there are no teachings for handling or dispensing a single unit dose of a drug that has high sensitivity to oxygen.

### SUMMARY

Applicant has discovered a means for dispensing a single unit-dose of an oxygen-sensitive solid drug without exposing the remaining unit dosages to oxygen. The present invention provides a pharmaceutical packaging means for dispensing a single dose of an oxygen-sensitive drug that includes a plurality of unit doses of an oxygen-sensitive drug, a lid and a blister: wherein each unit dose of the plurality of unit doses is individually encapsulated between the lid and the blister by means of a sealable laminate (preferably a heat-sealable laminate) deposited on the lid; and an oxygen absorber is incorporated into the laminate, the blister, a coating interposed between the laminate and the lid, or a combination thereof such that the oxygen absorber removes at least a portion of the oxygen from the air surrounding the oxygen-sensitive drug. The removal of the oxygen in the air reduces or eliminates undesired oxidation of the oxygen-sensitive drug thus enhancing the shelf-life stability of the drug. Preferably, the oxygen-absorber maintains a level of oxygen in the air surrounding the oxygen-sensitive drug less than or equal to about 10.0%, more preferably less than or equal to about 5%, even more preferably less than or equal to about 1.0%, most preferably less than or equal to about 0.5% for 2 years.

In another embodiment of the present invention, a process is provided for manufacturing a pharmaceutical packaging means for dispensing a single dose of an oxygen-sensitive drug comprising the steps of:

(i) providing a blister having a plurality of recesses,

(ii) placing a single unit dose of an oxygen-sensitive drug inside each of the plurality of recesses in the blister, and

(iii) laminating onto the blister from step (ii) a lid comprising a backing having deposited thereon a sealable laminate and a thermoplastic layer containing an oxygen absorber interposed between the backing and the sealable laminate to produce a package containing a plurality of encapsulated single unit doses of the oxygen-sensitive drug.

Optionally, the laminating step is performed in an inert atmosphere (e.g., nitrogen blanket).

### Definitions

As used herein, the term “unit dose” or “unit dosage” refers to physically discrete units that contain a predetermined quantity of active ingredient calculated to produce a desired therapeutic effect.

The term “drug” refers to a pharmaceutically active ingredient(s) and any pharmaceutical composition containing the pharmaceutically active ingredient(s). Pharmaceutical compositions include formulations as well as medicaments (e.g., powders, softgels, lyophiles, suppositories, capsules and tablets, intended for ingestion, or other methods of entering the body for medical purposes either directly or by constitution with other materials including liquids followed by ingestion or injection into humans or animals).

The term “oxygen-sensitive” or “oxygen-sensitivity” refers to the ability of a substance to react with oxygen under normal ambient conditions. The reaction may involve the addition of oxygen to the substance, removal of a hydrogen from the substance, or the loss or removal of one or more electrons from a molecular entity, with or without concomitant loss or removal of a proton or protons.

The term “lid” refers to the backing or substrate component of a packaging construction. The substrate can be a plastic, a foil or a combination of materials including plastic or foil with paper (cardboard).

The term “blister” refers to a sheet in a package construction with recesses designed to hold dosage forms. The sheet may be a plastic, a foil, or combination thereof.

“Thermoforming” is a process wherein a thermoplastic sheet is deformed with heat and pressure to form a blister.

The term “plurality” refers to one or more.

### DETAILED DESCRIPTION

Although the use of oxygen absorbing sachets or cartridges in plastic or glass bottles can provide a significant increase in shelf-life, once the bottle is opened or the seal broken, the absorber will rapidly become depleted. For many drugs, the chemical stability is adequate for use during the limited time period after opening. However, for drug formulations that are particularly oxygen sensitive or are used by patients periodically over long periods of time, it is preferred to provide oxygen absorption capability on individual dosages. The most challenging are those formulations that are both oxygen and moisture sensitive. Applicants have discovered configurations of blister packaging that provide the absorption capacity to address these unmet needs. Blister packs are well-known in the packaging industry and are widely used for the packaging of pharmaceutical unit dosage forms such as tablets, capsules, and the like. In general, the blister pack includes a lid having deposited thereon a heat-seal, which is laminated to a blister. The term “lid” generally refers to a backing or substrate with coatings on it. The substrate can be plastic, foil or a combination of materials including plastic or foil with paper (cardboard). The lid can be deformable to allow for pressure push through of a dosage form, or it may require peeling of a laminated backing to allow for push through. The term “blister” generally refers to a substrate with recesses designed to hold dosage form. The substrate typically comprises a plurality of recesses (including a single recessed space). The recesses can be preformed in a theromforming process or be made by deforming a substrate onto a dosage form. The blister can be made from plastic materials, including multilayers, or from foils. The blister is usually a relatively stiff material, preferably transparent, and may optionally contain a colorant.

A laminate is typically deposited on the lid to allow for sealing between the lid and the blister thus encasing the dosage form in the packaging unit. The laminate can be applied to the lid by methods common in the packaging industry including coating, extruding and lamination. A preferred laminate is a heat-sealable laminate (e.g., thermoplastic coating or thin pressure-sensitive adhesive coating (i.e., having a thickness from about 0.5 μm to about 15 μm)). Though the invention describes the use of a heat-seal where lamination occurs at some elevated temperature, it will be appreciated by those skilled in the art that the laminate could comprise other adhesive technologies, including pressure sensitive adhesives, photo-curing adhesives and two component (epoxy) adhesives.

A general review of blister packaging and its use in pharmaceutical packaging may be found in Pharm. Tech. November, pp. 68-78 (2000). Generally, the tablets or capsules are placed in the recesses of the blister and then the lid is laminated to it thus sealing the blister to encapsulate the tablets or capsules. Optionally, the lamination can be performed in an inert atmosphere (e.g., nitrogen blanket), though this is expensive and generally does not lead to very low oxygen head-space levels.

In one embodiment of the present invention, the strength of the lid is such that the tablets or capsules can be removed from the blister pack by manually applying pressure on the blister recesses whereby an opening is formed in the foil at the place of the recess. The tablet(s) or capsule(s) can then be removed through the opening. Alternatively, the lid may be peeled away from the blister thus exposing the tablet(s) or capsule(s) for easy removal. In some cases (e.g., a tamper-proof construction), a paper, cardboard or plastic backing is placed over the lid which is removed before the lid can be ruptured. The additional backing also provides a surface for printing information such as the trademark of the encapsulated drug.

The surface area of the plastic significantly increases the potential for oxygen permeation. Even when packaged anaerobically, oxygen permeation can quickly replace the inert atmosphere. To mitigate this effect, blister-packaging materials have evolved to minimize oxygen permeation. In addition, materials which have good oxygen barrier properties are often undesirable from an environmental perspective. These materials include such halogenated plastics as poly(vinylchloride) and poly(vinylidine chloride). In practice, only modest reductions in oxygen levels are observed and maintained with blister packaging even with foil-foil blisters which have virtually no permeability to oxygen due to the challenges of truly packaging anaerobically. The present invention provides for the introduction of an oxygen absorber into the packaging construction to eliminate and/or reduce exposure of the drug to oxygen. To be effective, the oxygen-absorber is incorporated into the construction such that the air surrounding the oxygen-sensitive drug is in direct or indirect (i.e., with an oxygen permeable material positioned between the air surrounding the oxygen-sensitive drug and the oxygen absorber) contact with the oxygen-absorber in a sufficient amount for the oxygen-absorber to remove at least a portion of the oxygen from the air to stop or retard the degradation process. The amount of oxygen-absorber added will depend upon the volume of air surrounding the drug, the anticipated permeation of oxygen through the blister, the oxidation potential of the drug, and the means by which the oxygen-absorber is incorporated into the construction. The oxygen-absorber need not remove 100% of the oxygen from the air; however, the absorber should be capable of maintaining a level of oxygen less than or equal to about 10.0%, more preferably less than or equal to about 5%, even more preferably less than or equal to about 1.0%, and most preferably less than or equal to about 0.5% for 2 years.

One means for introducing the oxygen-absorber involves the placement of the oxygen-absorbing system in the lid. Preferably, the absorber is embedded in a second thermoplastic layer which is co-extruded (or coated) with the laminate onto the lid. Any process for incorporating additives into a thermoplastic material prior to extrusion may be used to incorporate the absorber and is well known to those skilled in the art. For example, the absorber may be milled into the resin which is then extruded or simply dispersed or solubilized in a solvent and then coated onto a substrate. Another means of introducing the oxygen-absorber involves incorporating the oxygen absorber directly into the laminate.

Another means of introducing the oxygen-absorber involves placement of the absorber onto the blister. In a preferred embodiment, this entails co-extrusion of the oxygen absorber with a barrier material. In a more preferred embodiment, a trilayer co-extruded film can be formed wherein the absorbing plastic is sandwiched between a barrier layer (on the outside) and an oxygen permeable layer on the inside. This oxygen permeable layer serves to prevent direct physical and chemical contact of the dosage form with the oxygen absorbing material and any products it produces. This is especially desirable for oxygen absorbing materials that are not deemed to be safe for direct pharmaceutical contact by regulatory bodies. A preferred barrier material is a plastic having low oxygen permeability. Suitable materials include polyvinylchloride (PVC), polyvinylalcohol (PVOH), ethylenevinylalcohol (EVOH) and polyvinylidinechloride (PVDC). Preferably, the oxygen barrier polymer has a thickness between about 10 μm and about 300 μm, more preferably, between about 100 μm and about 200 μm. For those embodiments where moisture and oxygen barrier properties are desired, the barrier layer may contain a co-extrusion of materials, one with good oxygen barrier properties and the other with good moisture barrier properties. Since the oxygen barrier properties are often affected adversely by moisture, the moisture barrier material is preferably positioned on the outside of the oxygen barrier material (followed by the oxygen absorbing material). Preferably, the co-extruded layers of barriers and absorbing materials are thermoformable to enable flexible manufacturing of the blister.

In another embodiment of the present invention, the blister uses a metal as the barrier material. For example, the construction may consist of a foil (such as aluminum) with a coating or lamination of the oxygen absorbing material, with an optional second coating or lamination (or co-extrusion) of an oxygen permeable barrier material to avoid contact of the dosage form with the oxygen absorbing material or its degradants (or plasticizers). Alternatively, the metal barrier can be formed by deposition of a metal onto the oxygen absorbing plastic, such as by vacuum deposition.

If a water-initiated oxygen-absorber is used, then a sufficient amount of moisture to initiate the oxidation process is introduced prior to sealing the lid to the blister. This may be achieved by controlled water addition (humidity exposure) before or during packaging. Suitable water-initiated, oxygen-absorbers include metal-based absorbers such as particulate-type iron (e.g., hydrogen reduced iron, electrolytically reduced iron, atomized iron, and milled pulverized iron powders), copper powder, and zinc powder. A preferred metal-based absorber is an iron powder. A moisture-holding material may be incorporated with the absorber to provide a self-activated system. Suitable moisture-holding materials include activated carbon, silicas, zeolites, molecular sieves, hydrogels, and diatomaceous earth. The particular moisture-holding materials used will depend upon the humidity level of the environment. For example, in a very low humidity environment, a moisture carrying material such as a hydrogel that partially binds water may be preferred rather than a simple moisture absorbent (or desiccant). An accelerator may also be incorporated such as a metallic iodide or bromide as described in U.S. Pat. No. 6,133,361, incorporated herein by reference. An example of a suitable thermoplastic resin containing an oxygen absorber is Amosorb™ 3000 (available from BP Amoco Chemicals). Other resins appropriate for the current invention include those made using ascorbic acid or other easily oxidized organic compounds.

A preferred oxygen absorbing material is an absorber activated by ultraviolet-light. The UV-photo-activated absorber may be activated by exposing the absorber to UV light immediately before insertion of the dosages into the packaging, or in some cases, by exposure to UV light through the blister itself after sealing with the drug. This last approach assumes that the blister is sufficiently transparent to the UV light to allow activation of the absorber and the drug is stable to the light exposure. Suitable UV-activated oxygen absorbers are described in US Patent Nos. 6,139,770 and 6,057,013, incorporated herein by reference. It will be appreciated by those skilled in the art that the oxygen absorbing material may be compounded with other materials (such as polymers and plasticizers) in order to render the resulting blend co-extrudable with the other materials as part of the construction. For optimization, properties such as extrudability, adhesion and thermoformability are generally considered. The amount of absorbing resin used typically depends on the absorption capacity, the oxygen head-space, the oxygen permeation rate and the desired shelf-life. The preferred thickness of the oxygen absorbing layer is between about 5 μm and about 100 μm, more preferred between about 10 μm and about 30 μm. In a preferred embodiment, the configurations involve using an ultraviolet photo-activated oxygen absorber is incorporated either beneath the laminate on the lid or as a co-extruded material as part of the blister. The photo-activated oxygen absorber is typically activated prior to sealing the drug into the blister package. Other activation methods can also be employed. Suitable methods include electron beam, gamma irradiation and microwave treatment. It will be appreciated by those skilled in the art that activation enables the processing (extrusion, molding or coating) and storage of the resin and package in air without oxygen scavenging prior to final packaging with the pharmaceutical. As such, any activation mechanism which effectively switches on the oxygen absorbing ability of the system at the appropriate time (generally immediately before or after the drug is sealed in the unit dose package) will be effective in the practice of the present invention.

Since the protection of the dosage form from environmental oxygen will require consumption of the oxygen absorbing material, for a fixed amount of absorber, there will be a limited shelf-life. To increase the shelf-life without increasing the thickness, complexity or cost of the blister package, it can be desirable to include secondary packaging as part of the overall packaging. Such secondary packaging preferentially consists of heat-sealed pouches containing one or more “cards” of blisters. This pouch can be a plastic or foil. Still more preferred is that an oxygen absorbing sachet or cartridge (for example, AgelessTM made by Mitsubishi Gas Co., or Fresh PaxTM by Multisorb Corp.) be incorporated into the pouch. In typical use, the patient will open the pouch and consume the tablets of the blister card within a fixed period (e.g., 30-90 days).

The packaging construction of the present invention may be used for the distribution of any pharmaceutical drug; however, it is especially useful for oxygen-sensitive drugs. Any pharmaceutical composition that may degrade as a result of exposure to oxygen may be incorporated into the inventive packaging construction. Some examples of oxygen-sensitive materials which are subject to degradation due to oxygen exposure include materials such as amines either as salts or as free bases, sulfides, allylic alcohols, phenols and the like. In particular, pharmaceutically active compounds or materials which benefit by the present invention include basic drugs with pKa values in the range from about 1 to about 10, more particularly in the range from about 5 to about 9. Also benefiting from the present invention are pharmaceutically active compounds or materials having redox potentials less than or equal to about 1300 mV versus Ag/Ag⁺, more preferably less than or equal to about 1000 mV versus Ag/Ag⁺. Although many drugs exist for which either of these functional groups or redox potentials criteria are met and yet are stable to oxygen, few drugs outside these specifications are oxygen sensitive. Examples of some specific pharmaceutically active compounds that might benefit from the application of the packaging means of the present invention include compounds such as pseudoephedrine, tiagabine, acitretin, rescinnamine, lovastatin, tretinoin, isotretinoin, simvastatin, ivermectin, verapamil, oxybutynin, hydroxyurea, selegiline, esterified estrogens, tranylcypromine, carbamazepine, ticlopidine, methyidopahydro, chlorothiazide, methyldopa, naproxen, acetominophen, erythromycin, bupropion, rifapentine, penicillamine, mexiletine, verapamil, diltiazem, ibuprofen, cyclosporine, saquinavir, morphine, sertraline, cetirizine, N-[[2-methoxy-5-(1-methyl)phenyl]methyl]-2-(diphenylmethyl)-1-azabicylco[2.2.2]octan-3-amine and the like.

The present invention can also stabilize excipients in the dosage form to oxidative degradation (e.g., degradation that leads to discoloration, harmful reactivity with the pharmaceutical agent or changes in the dosage form performance, such as dissolution or disintegration rates). Nonexclusive examples of excipients commonly used in pharmaceutical formulations that could be stabilized by application of the present invention include poly(ethylene oxides), poly(ethylene glycols) and poly(oxyethylene) alkyl ethers. The present invention provides for the stabilization of pharmaceutical dosages to oxidation. The degree to which the stabilization occurs can be assessed by spectroscopy (light absorption or reflection) and/or by spectroscopic means. A particularly preferred means for characterization involves the use of HPLC. The present invention need not completely eliminate degradation and or discoloration to be effective; however, preferably degradation and/or discoloration of the oxygen-sensitive drug versus samples packaged without an oxygen absorber is reduced by at least about 20%, more preferably by about 50% and most preferably by about 75%.

What is claimed is:

1. A pharmaceutical packaging means for dispensing a single dose of an oxygen-sensitive drug comprising a plurality of unit doses of an oxygen-sensitive drug, a lid and a blister: wherein each unit dose of said plurality of unit doses is individually encapsulated between said lid and said blister by means of a sealable laminate deposited on said lid; and an oxygen absorber is incorporated into said laminate, said blister, said lid, a layer interposed between said laminate and said lid, or a combination thereof such that said oxygen absorber removes at least a portion of oxygen from the air surrounding said oxygen-sensitive drug.

2. The pharmaceutical packaging means of claim 1 wherein said oxygen absorber is incorporated into said layer interposed between said laminate and said lid.

3. The pharmaceutical packaging means of claim 1 wherein said oxygen absorber is incorporated into both said blister and said layer interposed between said laminate and said lid.

4. The pharmaceutical packaging means of claim 1, 2 or 3 wherein said oxygen absorber is selected from the group consisting of a moisture-activated absorber, a self-activated absorber, a UV-activated absorber, an electron beam activated absorber, a radiation activated absorber, a microwave activated absorber and combinations thereof.

5. The pharmaceutical packaging means of claim 4 wherein said oxygen absorber is a moisture-activated absorber selected from the group consisting of a particulate-type iron, a copper powder, and a zinc powder.

6. The pharmaceutical packaging means of claim 5 wherein said oxygen absorber is a particulate-type iron selected from the group consisting of a hydrogen reduced iron, an electrolytically reduced iron, an atomized iron, and a milled pulverized iron powder.

7. The pharmaceutical packaging means of claim 1 wherein the oxygen content of the air surrounding said oxygen-sensitive drug is maintained at a level less than or equal to about 10.0% for about two years.

8. The pharmaceutical packaging means of claim 1 wherein the oxygen content of the air surrounding said oxygen-sensitive drug is maintained less than or equal to about 5.0% for about two years.

9 The pharmaceutical packaging means of claim 1 wherein the oxygen content of the air surrounding said oxygen-sensitive drug is maintained at a level less than or equal to about 1.0% for about two years.

10. The pharmaceutical packaging means of claim 1 wherein the oxygen content of the air surrounding said oxygen-sensitive drug is maintained at a level less than or equal to about 0.5% for about two years.

11. The pharmaceutical packaging means of claim 1 wherein said oxygen-sensitive drug comprises a pharmaceutically active ingredient selected from the group consisting of amines, phenols, sulfides and allylic alcohols.

12. The pharmaceutical packaging means of claim 1 wherein said oxygen-sensitive drug comprises an oxygen sensitive excipient.

13. The pharmaceutical packaging means of claim 1 wherein said oxygen-sensitive drug comprises an oxygen-sensitive pharmaceutically active compound.

14. The pharmaceutical packaging means of claim 13 wherein said oxygen-sensitive pharmaceutically active compound is a basic drug having a pKa value from about 1 to about 10.

15. The pharmaceutical packaging means of claim 13 wherein said oxygen-sensitive pharmaceutically active compound is a basic drug having a pKa value from about 5 to about 9.

16. The pharmaceutical packaging means of claim 13 wherein said oxygen-sensitive pharmaceutically active compound has a redox potential less than or equal to about 1300 mV.

17. The pharmaceutical packaging means of claim 13 wherein said oxygen-sensitive pharmaceutically active compound has a redox potential less than or equal to about 1000 mV.

18. The pharmaceutical packaging means of claim 13 wherein said oxygen-sensitive pharmaceutically active compound is selected from the group consisting of pseudoephedrine, tiagabine, acitretin, rescinnamine, lovastatin, tretinoin, isotretinoin, simvastatin, ivermectin, verapamil, oxybutynin, hydroxyurea, selegiline, esterified estrogens, tranylcypromine, carbamazepine, ticlopidine, methyldopahydro, chlorothiazide, methyidopa, naproxen, acetominophen, erythromycin, bupropion, rifapentine, penicillamine, mexiletine, verapamil, diltiazem, ibuprofen, cyclosporine, saquinavir, morphine, sertraline, cetirizine, and N-[[2-methoxy-5-(1-methyl)phenyl]methyl]-2-(diphenylmethyl)-1-azabicylco[2.2.2]octan-3-amine.

19. The pharmaceutical packaging means of claim 1 wherein degradation or discoloration of said oxygen sensitive drug is reduced by at least about 20%.

20. The pharmaceutical packaging means of claim 1 wherein degradation or discoloration of said oxygen sensitive drug is reduced by at least about 50%.

21. The pharmaceutical packaging means of claim 1 wherein degradation or discoloration of said oxygen sensitive drug is reduced by at least about 75%.

22. A process for manufacturing a pharmaceutical packaging means for dispensing a single dose of an oxygen-sensitive drug comprising the steps of:
(i) providing a blister having a plurality of recesses,
(ii) placing a single unit dose of an oxygen-sensitive drug inside each of said plurality of recesses in said blister, and
(iii) laminating onto said blister from step (ii) a lid comprising a backing having deposited thereon a sealable laminate and a thermoplastic layer containing an oxygen absorber interposed between said backing and said sealable laminate to produce a package containing a plurality of encapsulated single unit doses of said oxygen-sensitive drug.

23. The process of claim 22 wherein said laminating step (iii) is preformed in an inert atmosphere.

24. The process of claim 22 wherein said oxygen absorber is selected from the group consisting of a moisture-activated absorber, a self-activated absorber, a UV-activated absorber and combinations thereof.

25. The process of claim 24 wherein said oxygen absorber is a moisture-activated absorber selected from the group consisting of a particulate-type iron, a copper powder, and a zinc powder.

26. The process of claim 25 wherein said oxygen absorber is a particulate-type iron selected from the group consisting of a hydrogen reduced iron, an electrolytically reduced iron, an atomized iron, and a milled pulverized iron powder.

27. The process of claim 22 wherein said oxygen-sensitive drug comprises a pharmaceutically active ingredient selected from the group consisting of amines, phenols, sulfides and allylic alcohols.

28 The process of claim 22 wherein said oxygen-sensitive drug comprises an oxygen sensitive excipient.

29. The process of claim 22 wherein said oxygen-sensitive drug comprises an oxygen-sensitive pharmaceutically active compound.

30. The process of claim 29 wherein said oxygen-sensitive pharmaceutically active compound is a basic drug having a pKa value from about 1 to about 10.

31. The process of claim 29 wherein said oxygen-sensitive pharmaceutically active compound is a basic drug having a pKa value from about 5 to about 9.

32. The process of claim 29 wherein said oxygen-sensitive pharmaceutically active compound has a redox potential less than or equal to about 1300 mV.

33. The process of claim 29 wherein said oxygen-sensitive pharmaceutically active compound has a redox potential less than or equal to about 1000 mV.

34. The process of claim 29 wherein said oxygen-sensitive pharmaceutically active compound is selected from the group consisting of pseudoephedrine, tiagabine, acitretin, rescinnamine, lovastatin, tretinoin, isotretinoin, simvastatin, ivermectin, verapamil, oxybutynin, hydroxyurea, selegiline, esterified estrogens, tranylcypromine, carbamazepine, ticlopidine, methyidopahydro, chlorothiazide, methyldopa, naproxen, acetominophen, erythromycin, bupropion, rifapentine, penicillamine, mexiletine, verapamil, diltiazem, ibuprofen, cyclosporine, saquinavir, morphine, sertraline, cetirizine, and N-[[2-methoxy-5-(1-methyl)phenyl]methyl]-2-(diphenylmethyl)-1-azabicylco[2.2.2]octan-3-amine.
